(19) United States
(12) Patent Application Publication
(10) Pub. No.: US 2015/0164485 A1
(43) Pub. Date: Jun. 18, 2015

(54) DNA MEDICATED COLLECTION KITS

(71) Applicant: ASCLEMED USA, INC. dba EnovaChem Manufacturing, Torrance, CA (US)

(72) Inventor: Robert Prentiss Nickell, Torrance, CA (US)

(73) Assignee: ASCLEMED USA, INC. dba EnovaChem Manufacturing, Torrance, CA (US)

(21) Appl. No.: 14/562,555

(22) Filed: Dec. 5, 2014

Related U.S. Application Data
(60) Provisional application No. 61/914,958, filed on Dec. 12, 2013.
(60) Provisional application No. 61/932,721, filed on Jan. 28, 2014.
(60) Provisional application No. 62/072,286, filed on Oct. 29, 2014.

(51) Int. Cl. A61B 10/00; A61M 19/00; B65D 5/64

(52) U.S. Cl. CPC A61B 10/0051; B65D 5/64; A61B 10/007; A61B 10/0096; A61M 19/00; A61B 2010/0216

(57) ABSTRACT

Provided are kits for collecting samples from a person for DNA (Deoxyribonucleic acid) testing. The kits can optionally also allow for collection of a urine sample. Components of the kits can include lid local anesthetic such as lidocaine hydrochloride, an item that allows for cleaning the mouth, such as mouthwash, a tool for removing DNA such as buccal swabsticks, a container for collecting urine, such as a urine cup, a pair of gloves, a face mask, and a CSR wrap. DNA samples are collected from the buccal area by scraping the area with a buccal swabstick provided in the kit.

### CROSS-REFERENCE

The present application claims the benefit of U.S. provisional Appl. No. 61/914,958, filed on Dec. 12, 2013, U.S. provisional Appl. No. 61/932,721, filed on Jan. 28, 2014, U.S. provisional Appl. No. 62/072,286, filed on Oct. 29, 2014, all of which are incorporated herein by reference in their entirety.

### BACKGROUND SECTION OF THE INVENTION

With the changing reimbursements, types of health care insurance, and new regulations with regards to treatment, a medical practitioner and his/her team when treating a patient is under immense time pressures to perform quickly, efficiently and with safety for the patient. The medical practitioner can lose valuable time trying to not only locate the necessary drugs and materials to treat the patient but as well to insure the compliance, the safety of the patient by preventing contamination. In some instances, not all the drugs and materials may be readily available, in the current exam room, resulting in delayed treatment and possible and potential medical errors. In addition, the medical practitioner or his/her team may make a mistake by incorrectly choosing the drug, by incorrectly identifying the product, not checking the expiration date, not returning it to the proper environment for storage, or not having enough product. Additionally, a DNA analysis by itself is limited in the kind of information that it can provide about a patient.

There is a need in the art for a convenience kit that solves the above mentioned problems particularly in the field of DNA collection from patients and can provide additional information about a patient not possible solely with a DNA analysis.

### SUMMARY SECTION OF THE INVENTION

Provided is a kit comprising a first container and following components placed inside the first container: a) a second container for collection of a urine sample; and b) a tool for collection of cells for DNA (Deoxyribonucleic acid) testing from a person's buccal area. The tool can be one or more buccal swabsticks. The kit can further comprise a local anesthetic. The local anesthetic can be lidocaine hydrochloride. The kit can further comprise one or more components for cleaning the buccal area. The one or more components for cleaning the buccal area can be selected from the group consisting of one or more of mouthwash, floss, to toothbrush, and combinations thereof. The kit can further comprise a soothing agent. The soothing agent can be glycerin in form of a glycerin swab. The first container can be a box with a lid. The kit can further comprise one or more of gloves, face mask and CSR (Central Supply Room) wrap placed inside of the first container. The kit can comprise: a) lidocaine hydrochloride oral topical solution; b) mouthwash; c) a glycerin swab; d) a buccal swabstick; e) a sterile urine container; f) a pair of gloves; g) a face mask; and h) a CSR (Central Supply Room) wrap. The kit can comprise: a) lidocaine hydrochloride oral topical solution with a 2% concentration; b) a fourth container of mouthwash; c) a plurality of glycerin swabs; d) a plurality of buccal swabsticks; c) a sterile urine cup; f) a pair of gloves; g) a face mask; and h) a CSR (Central Supply Room) wrap;

Provided is a kit comprising a first container and following components placed therein: a) a first tool for collection of cells for DNA (Deoxyribonucleic acid) testing from a person's buccal area; b) a local anesthetic; and c) a soothing agent. The first tool can be one or more buccal swabsticks. The local anesthetic can be lidocaine hydrochloride. The soothing agent can be one or more of glycerin swabs. The kit can further comprise one or more items for cleaning the buccal area. The one or more components for cleaning the buccal area can be selected from the group consisting of one or more of mouthwash, floss, toothbrush, and combinations thereof. The kit can comprise one or more of gloves, face mask and CSR (Central Supply Room) wrap. The kit can comprise a) lidocaine Hydrochloride; b) mouthwash; c) a f glycerin swab; d) a buccal swabstick; e) a toothbrush; f) a pair of gloves; g) a face mask; and h) a csr (Central Supply Room) wrap. The kit can comprise: a) lidocaine hydrochloride oral topical solution with a 2% concentration; b) mouthwash; c) a plurality of oral glycerin swabs; d) a plurality of buccal swabsticks; e) a toothbrush; f) a pair of gloves; g) a face mask; and h) a CSR (Central Supply Room) wrap. The kit can comprise: a) Lidocaine HCl; b) a glycerin swab; c) a buccal swabstick; d) a pair of gloves; c) a face mask; and f) a CSR (Central Supply Room) % Tap. The kit can comprise: a) lidocaine hydrochloride oral topical solution with a 2% concentration; b) a plurality of oral glycerin swabs; c) a plurality of buccal swabsticks; d) a pair of gloves; e) a face mask; and t) a CSR (Central Supply Room) wrap.

Provided is a kit comprising a) one or more components for collecting a sample (such as cells) for DNA analysis from buccal (cheek/mouth) area of a person; b) one or more components for collecting a patient's urine. The DNA can be collected with buccal swabsticks. The urine can be collected with a container such as a urine cup. The kit can further comprise one or more of lidocaine hydrochloride oral topical solution, mouthwash, gloves, face mask and CSR wrap. The urine can be used to test for presence of any active pharmaceutical ingredient (or metabolite) such as those that are listed below, including clopidogrel. The sample can be tested for genes that regulate one or more of P450 CYP2C19, CYP2C9 and CYP2D6 activity. The dose of the drug can subsequently be adjusted according to how a patient metabolizes a drug and the genes that the person has. The kit can also be used to take a DNA sample for testing to determine a Genetic Addiction Risk Score (GARS) and a sample of urine for presence and/or amount of one or more of active pharmaceutical ingredient (or metabolite).

Provided is a kit comprising a holder (typically a box) and following components placed inside the holder: a) a container of lidocaine HCl suitable for oral topical administration; b) mouthwash; c) toothbrush or floss string; d) swabsticks; e) oral glycerin swabs; f) gloves; g) face mask; and h) CSR wrap.

Each component of the kit can be placed in an individual package or a third container. Typically the mask does not have a wrapping.

### BRIEF DESCRIPTION OF THE FIGURES

FIG. 1 illustrates a box for holding components of the kit.

FIG. 2 illustrates a glove.

FIG. 3 illustrates a face mask.

FIG. 4 illustrates an instruction sheet.

FIG. 5 illustrates a CSR wrap.

FIG. 6 illustrates a swabstick and packaging for such swabstick.

FIG. 7 illustrates a urine cup or container.

FIG. 8 illustrates a toothbrush.

FIG. 9 illustrates a bottle.

FIG. 10 illustrates a sealed cap with a swab having a DNA sample. The stick is discarded.

FIG. 11 illustrates a piece of floss

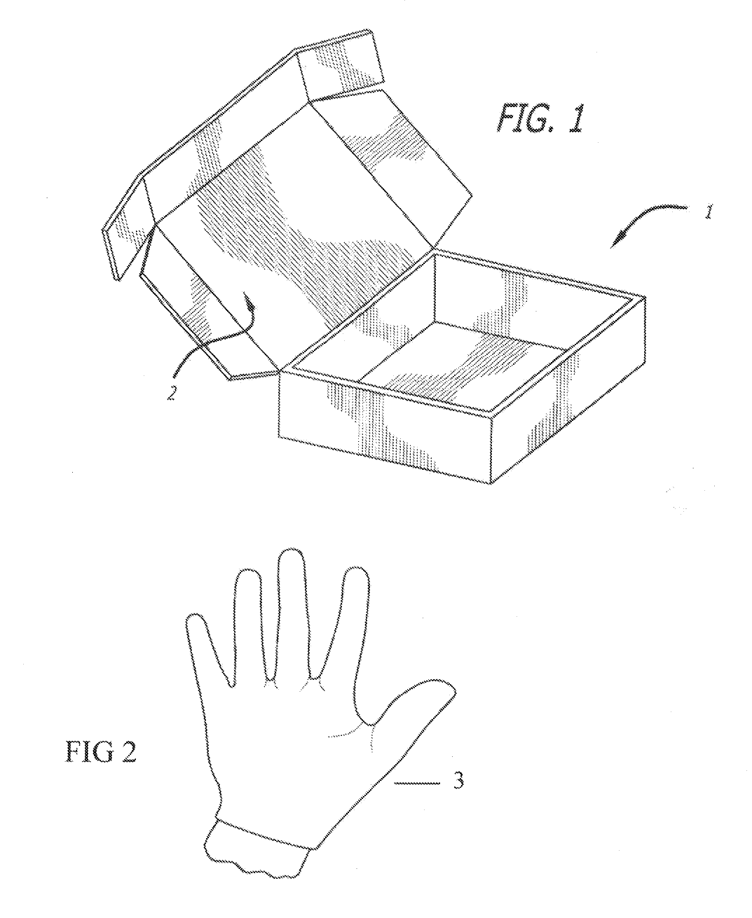

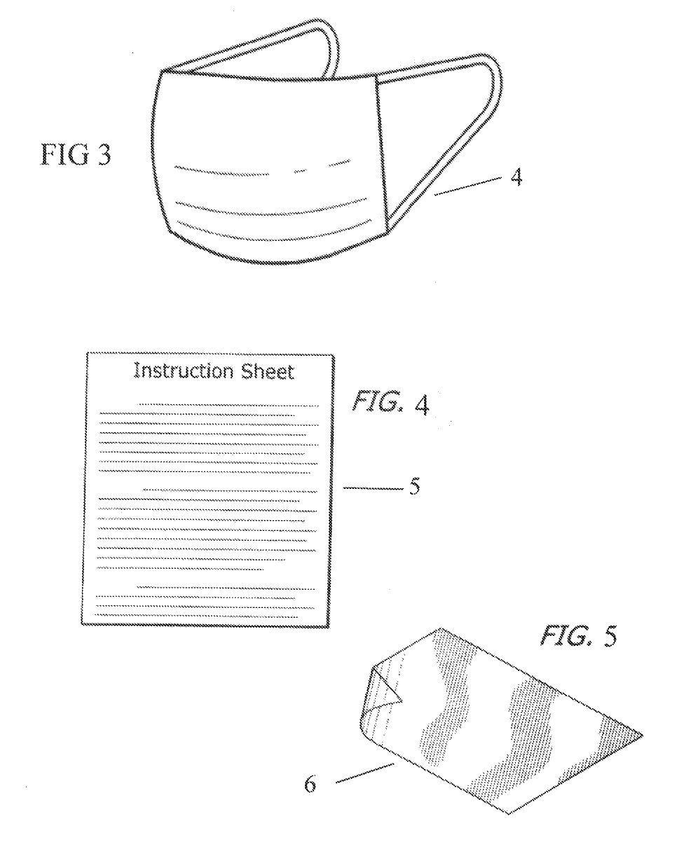

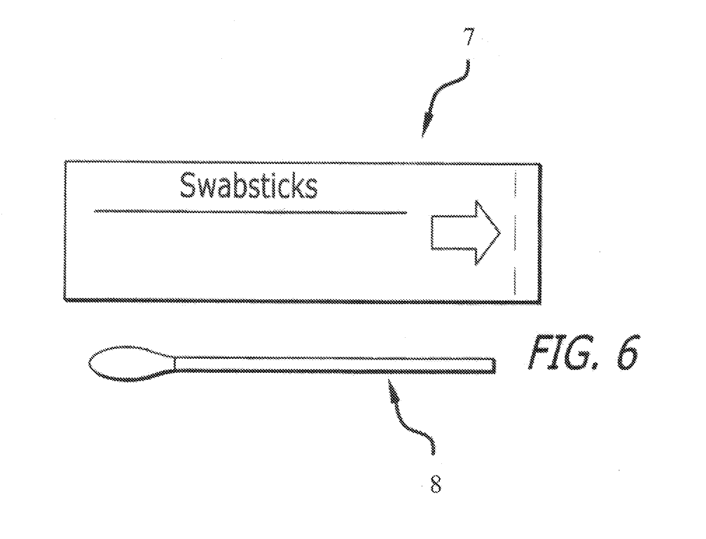

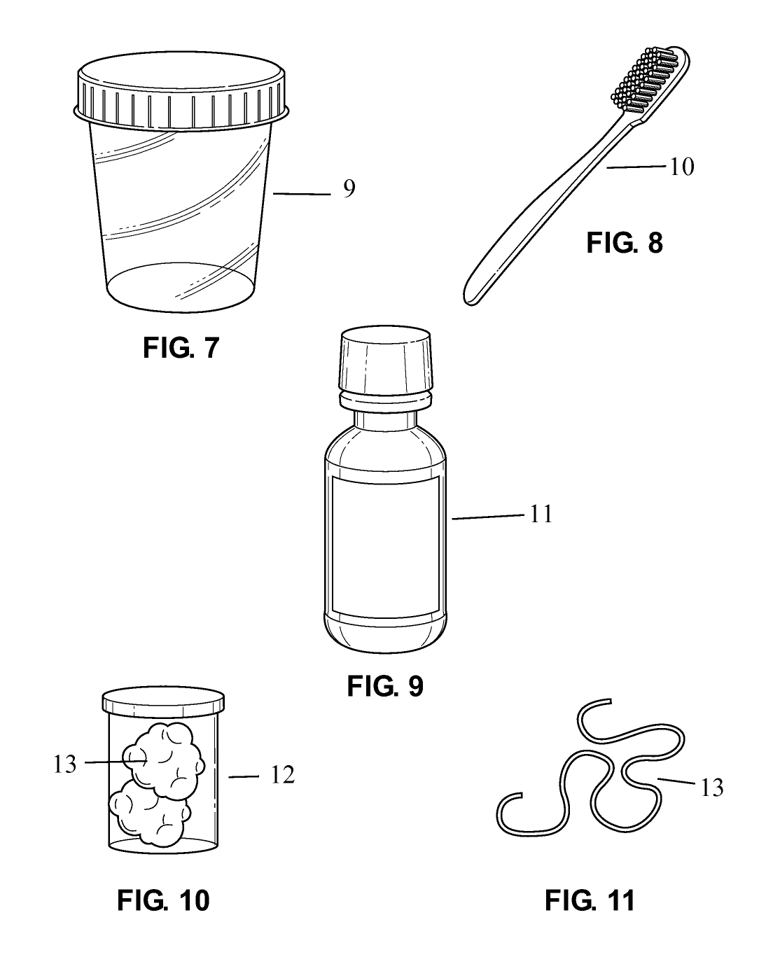

### DETAILED DESCRIPTION OF THE INVENTION

The present invention provides for kits that allow for taking of a specimen from the buccal area of a person for DNA testing. In one embodiment, the kit allows for taking samples of cells from the buccal area and urine for testing both the amount of a drug (and/or its metabolites) in a patient's urine and the person's genetic disposition as to how the person metabolizes that particular drug.

The kit can include a tool for collecting cells for DNA analysis from a person's cheek (buccal area). The tool is typically one or more of buccal swabsticks 8. The buccal swabsticks 8 are firmly pressed against the inside of a patient's cheek and can be used to scrape the cheek by moving the swab up and down as well as backwards and forwards. The swabsticks 8 can be in a packet 7 and can include a cap 12. After use, the swab 13 containing DNA cells in placed and sealed in the cap 12 and the stick is discarded. The cap 12 containing the swab is sent to a laboratory for DNA testing.

The kit can include both a urine cup 9 and buccal swabsticks 8. The urine cup 9 (container) allows for collecting urine for analysis of how a patient has metabolized the drug (such as rate of metabolism, action of metabolism), and the buccal swabsticks 8 for collecting cells for DNA analysis from a person's cheek (buccal area). The dose of the active pharmaceutical ingredient given to a patient can subsequently be adjusted based on the urine and the DNA analysis.

One component of the kit can allow a person to clean his or her mouth before collection of a sample from the buccal area. This component can be a mouthwash, for example Listerine® mouthwash, which can be in a bottle 11. By rinsing his or her mouth with mouthwash, the patient removes food particles and other contaminants that may interfere with DNA testing. Similarly, the kit can contain a short string of floss or a toothbrush (pasted or un-pasted). Any combination of one or more of mouthwash, floss 13, and toothbrush 10 can be placed in the kit.

One component of the kit can be a local anesthetic such as lidocaine hydrochloride, typically in the form of an oral topical solution. Lidocaine HCl is a local anesthetic that a patient can use to rinse the buccal area to reduce soreness left after a sample is taken from the cheek. The lidocaine HCl can be in an amount of about 2 to about 30 ml, such as about 20 ml. The lidocaine HCl can be in the form of an oral topical solution 2% USP that is viscous. The lidocaine HCl is placed in its own container, typically a bottle 9. A patient swishes and spits the lidocaine HCl.

One component of the kit can be one or more oral glycerin swabs 8. Glycerin is a soothing agent. Glycerin dabbed on the buccal area can help soothe the area from which samples are taken.

One component of the kit can be a urine cup 9. The urine cup 9 (or any container) can be used to collect a sample of urine from a patient. The urine cup 9 can be in a sterile plastic wrapping or a box.

One component of the kit can be a glove 3. The gloves 3 can be any suitable glove 3 that is used by a medical practitioner, such as latex or nitrile gloves 3. In one embodiment, a pair of nitrile gloves 3 are part of the kit. Nitrile gloves are a type of disposable glove made of nitrile rubber, also known as Buna-N, Perbunan, or NBR. Nitrile gloves contain no latex. A medical practitioner can tear a package containing the gloves and wear the gloves before collection to help prevent contamination and increase compliance. The gloves 3 can be in a sterile plastic wrapping or a box.

One component can be a mask 4 to help prevent the spread of disease between the patient and the practitioner. The face mask 4 can be placed inside the kit without wrapping.

One component of the kit can be a CSR (Central Supply Room) wrap 6. The CSR wrap 6 can be put on a tray or a surface before placement of the other components of the kit. The CSR wrap 6 keeps a clean environment. The CSR wrap 6 can be in a plastic wrapping or a box.

The kit can comprise a box 1 in which the above mentioned components are placed. The box can be a rectangular cube 1 which has a lid 2 on top for opening the box.

The kit can contain an instruction sheet 5. The instruction sheet 5 can identify one or more components of the kit, provide instructions as to how to use each component, and optionally suggest an order of use for the components of the kit. The instruction sheet 5 can provide instructions as to where and how take a DNA sample. There can also be two instruction sheets 5, one for the patient (such as fir after care) and one for the medical practitioner.

Instead or in addition to an instruction sheet 5, each component of the kit can be described on the box 1 of the kit, for example on one or more sides of the box 1. Each component can have its own separate instruction sheet 5 provided by its own manufacturer.

The following table is a specific example of a kit used to take a urine sample and a DNA sample from a patient.

Kit #1

20 ml lidocaine hydrochloride oral topical solution 2% USP (Can be
2-30 ml) × 1
20 ml Listerine Mouthwash (can be 2-30 ml) × 1
1 pack of oral glycerin swabs (3 swabs/pack) (can be 1-3 individual
swabs)
3 buccal swabsticks
1 sterile urine cup
1 pair of gloves - size 7- (can be nitrile or latex)
1 face mask
1 csr wrap

The following tables are examples of kits without a urine cup:

Kit #2

20 ml lidocaine hydrochloride oral topical solution 2% USP × 1 (can
be 2-30 ml)
10 ml mouthwash × 1 (Can be 2-30 ml) (such as Listerine ® Zero)
1 pack of oral glycerin swabs (3 swabs/pack) or 1-3 individual
swabs
3 buccal swabsticks
1 toothbrush and/or short floss string (can be toothbrush only)
1 pair of gloves (latex or nitrile)
1 face mask
1 csr wrap

Kit #3

20 ml lidocaine hydrochloride oral topical solution 2% USP × 1 (can
be 2-30 ml)
1 pack of oral glycerin swabs (3 swabs/pack) or 1-3 individual
swabs
3 buccal swabsticks
1 pair of gloves (latex or nitrile)
1 face mask
1 CSR wrap

In a typical situation a medical practitioner opens the kit and puts on the gloves 3. The medical practitioner can also put on any face mask 4 and lay out the CSR wrap 5. The medical practitioner can place the items of the kit on the CSR warp 5 and/or use the CSR wrap 5 for placing materials that need to be discarded. The patient can be asked to clean their mouth, for example by being given a glass of water to rinse their mouth. If the kit contains mouthwash, toothbrush, or floss, these items can be provided to the patient to clean his or her mouth. The patient swishes the water and/or the mouth wash for at least 30 seconds. If the water is colored, the patient repeats the process until water is clear and colorless. This works to keep the sample free of contaminates and food particles that could affect sample processing. The medical practitioner opens the buccal swab packet and remove swab with attached cap from the collection tube. The patient is given the swabs. Alternatively, the medical practitioner can scrape the patient's cheek with the swab. The patient presses one flat side of the swab firmly against the inside of his/her cheek and begins to scrape moving the swab up and down as well as backwards and forwards. After 30 seconds, the swab can be turned over, pressing the other side of the swab against the inside of his/her cheek and scrape for an additional 30 seconds. This may cause some abrasion and bleeding may result. After the scraping, the patient's cheek feels textured or raw.

The medical practitioner then places the swab still on stick with attached cap 12 back into the collection tube. Holding tube and cap 12 together, the medical practitioner can pull the stick from the swab 8 and out through the cap of the collection tube. The plug of the cap is then closes, keeping the swab 8 inside. The stick can then be discarded. The step of collection with a swab 8 can be repeated an addition 2-6 times. If the patient has any discomfort, swelling or bleeding, the medical practitioner in consultation with a physician can utilize the glycerin swab sticks and/or the lidocaine HCl for the comfort and relief of the patient.

The DNA of a patient can be tested for different purposes, such as to confirm a family relationship, for crime-related purposes, for confirming identity of a person, or for diagnostic purposes. In one embodiment, the DNA of the person taken can be tested for various genetic factors, including for genes that regulate or affect CYP2C9, CYP2C19 and CYP2D6 activity. Cytochrome P450 2C19 (abbreviated CYP2C19), a member of the cytochrome P450 mixed-function oxidase system, is involved in the metabolism of xenobiotics in the body. It is involved in the metabolism of several important groups of drugs including many proton pump inhibitors and antiepileptics. In humnans, the CYP2C9 protein is encoded by the CYP2C19 gene. CYP2D6 a member of the cytochrome P450 mixed-function oxidase system, is one of the most important enzymes involved in the metabolism of xenobiotics in the body. In particular, CYP2D6 is responsible for the metabolism and elimination of approximately 25% of clinically used drugs. This enzyme also metabolizes several endogenous substances such as hydroxytryptamines and neurosteroids. CYP2C9 is an important cytochrome P450 enzyme with a major role in the oxidation of both xenobiotic and endogenous compounds.

The DNA of the person taken can also be used to determine a Genetic Addiction Risk Score (GARS). The GARS can be particularly useful with patients where a previous test for P450 CYP2C19, CYP2C9, and/or CYP2D6 activity shows that the patient has low metabolism and may be susceptible to forming an addiction due to low clearance (metabolism) of the drug. The DNA can for example be tested for dopaminergic genes (D1, D3, D4, and D5. DATA1, MAO, COMT). The genetic test can be then used to determine Genetic Addiction Risk Score (GARS). The GARS test can be particularly useful with patients where a previous test for P450 2C19, CYP2D16 and/or CYP2D6 activity shows that the patient has low metabolism and may be susceptible to forming an addiction due to low clearance (metabolism) of the drug. Chapter 19 of the following book is incorporated herein by reference for its teaching of gene testing to determine a GARS: “Gene Therapy Applications”, book edited by Chunsheng Kang, ISBN 978-953-307-541-9, Published: Aug. 23, 2011”

The urine of the patient can be tested for various drugs (active pharmaceutical ingredients) and their metabolites as provided below:

CYP2C9

| Class | |
|---|---|
| | Substrate |
| NSAID | diclofenac |
| | Ibuprofen |
| | Lomoxicam |
| | Meloxicam |
| | S-Naproxen−> |
| | Norpiroxicam |
| | Piroxicam |
| | Suprofen |
| Oral Hypoglycemic | Tolbutamide |
| agents | Glipizide |
| Angiotensin II Blockers | Losartan |
| | Irbesartan |
| Sulfonylureas | Glyburide |
| | Glibenclamide |
| | Glipizide |
| | Glimepiride |
| | Tolbutamide |
| Various Others | Amitriptyline |
| | Celecoxib |
| | Fluoxetine |
| | Fluvastatin |
| | Glyburide |
| | Nateglinide |
| | Phenytoin-4-OH2 |
| | Rosiglitazone |
| | Tamoxifen |
| | Torsemide |
| | S-Warfarin |
| | Inhibitors |
| Strong | Fluconazole |
| Medium | Amiodarone |
| Weak | Fenofibrate |
| Unknown relative Strength | Fluvastatin |
| | Flucvoxamine |
| | Isoniazid |
| | Lovastatin |
| | Phenylbutazone |
| | Probenacid |
| | Sertraline |
| | Sulfamethoxazole |
| | Sulfphenazole |
| | Teniposide |
| | Voriconazole |
| | Zafirlukast |

CYP2C19

| Class | |
|---|---|
| | Substrate |
| Proton Pump | Lansoprazole |
| Inhibitors | Omeprazole |
| | Pantoprazole |
| | Rabeprazole |
| Anit- Epileptics | Diazepam−> |
| | Norphenytoin |
| | S-Mephenytoin |
| | (Phenytoin) |
| | Phenobarbitone |
| Various Other | Amitriptyline |
| | Carisoprodol |
| | Citalopram |
| | Chloramphenicol |
| | Clomipramine |
| | Clopidigrel |
| | Cyclophosphamide |
| | Hexobarbital |
| | Imipramine N-DeME |
| | Indomethacin |
| | R-Mephobarbital |
| | Moclobemide |
| | Nelfinavir |
| | Nilutamide |
| | Primidone |
| | Progesterone |
| | Proguanil |
| | ProPranolol |
| | Teniposide |
| | R-Warfarin-->8-OH |
| | Inhibitors |
| Strong | Esomepracole |
| Medium | Lansopraxole |
| Weak | Omeprazole |
| Unknown relative Strength | Pantoprazole |
| | Rabeprazole |
| | Chloramphenicol |
| | Cimetidien |
| | Felbamate |
| | Fluoxetine |
| | Fluvoxamine |
| | Indomethacin |
| | Ketoconazole |
| | Modafinil |
| | Oxcarbazepine |
| | Probenicid |
| | Ticlopidine |
| | Topiramate |

CYP2D6

| Class | |
|---|---|
| | Substrate |
| Beta Blockers | Carvedilol |
| | S-Metoprolol |
| | Propafenone |
| | Timolol |
| Anit-Depressants | Amitriptyline |
| | Clomipramine |
| | Desipramine |
| | Fluoxetine |
| | Imipramine |
| | Paroxetine |
| | Venlafaxine |
| Anitpsychotics | Haloperidol |
| | Perphenazine |
| | Risperidone−> 9-OH |
| | Thioridazine |
| | Zuclopenthixol |
| Various Other | Alprenolol |
| | Amphetamine |
| | Aripiprazole |
| | Atomoxetine |
| | Bufuralol |
| | Chlorpheniramine |
| | Chlorpromazine |
| | Clonidine |
| | Codeine−> O-DesMe |
| | Debrisoquine |
| | Dexfenfluramine |
| | Dextromethorphan |
| | Dihydrocodeine |
| | Donepazil |
| | Duloxetine |
| | Encainide |
| | Flecainide |
| | Fluvoxamine |
| | Hyodrocodone |
| | Lidocaine |
| | Metoclopramide |
| | Methoxyamphetamine |
| | Mexiletine |
| | Minaprine |
| | Nebivolol |
| | Nortriptyline |
| | Ondansetron |
| | Oxycodone |
| | Perhexiline |
| | Phenacetin |
| | Phenformin |
| | Promethazine |
| | Propranolol |
| | Sparteine |
| | Tamoxifen |
| | Tramadol |
| | Inhibitors |
| Strong | Bupropion |
| | Cinacalecet |
| | Fluoxetine |
| | Paroxetine |
| | Quinidine |
| Medium | Duloxetine |
| | Sertraline |
| | Terbinafine |
| Weak | Amiodarone |
| | Cimetidine |
| Unknown relative Strength | Celecoxib |
| | Chlorpheniramine |
| | Chlorpromazine |
| | Citalopram |
| | Clemastine |
| | Clomipramine |
| | Cocaine |
| | Diphenhydramine |
| | Doxepin |
| | Doxorubicin |
| | Escitalopram |
| | Halofantrine |
| | Haloperidol |
| | Histamine H1 Receptor |
| | Antagonists |
| | Hydroxyzine |
| | Levomepromazine |
| | Methadone |
| | Metoclopramide |
| | Mibefradil |
| | Midodrine |
| | Moclobemide |
| | Perphenazine |
| | Ranitidine |
| | Reduced-haloperidol |
| | Ritonavir |
| | Ticlopidine |
| | Tripelennamine |

What is claimed is:

1. A kit comprising a first container and following components placed inside the first container:
a) a second container for collection of a urine sample; and
b) a tool for collection of cells for DNA (Deoxyribonucleic acid) testing from a person's buccal area.

2. The kit of claim 1, wherein the tool is one or more buccal swabsticks.

3. The kit of claim 1, further comprising a local anesthetic.

4. The kit of claim 3, wherein the local anesthetic is lidocaine hydrochloride.

5. The kit of claim 1, further comprising one or more components for cleaning the buccal area.

6. The kit of claim 5, wherein the one or more components for cleaning the buccal area is selected from the group consisting of one or more of mouthwash, floss, toothbrush, and combinations thereof.

7. The kit of claim 1, further comprising a soothing agent.

8. The kit of claim 7, wherein the soothing agent is glycerin in form of a glycerin swab.

9. The kit of claim 1, wherein the first container is a box with a lid.

10. The kit of claim 1, further comprising one or more of gloves, face mask and CSR (Central Supply Room) wrap placed inside of the first container.

11. The kit of claim of 1, wherein the kit comprises:
a) lidocaine hydrochloride oral topical solution;
b) mouthwash;
c) a glycerin swab;
d) a buccal swabstick;
e) a sterile urine container;
f) a pair of gloves;
g) a face mask; and
h) a CSR (Central Supply Room) wrap.

12. The kit of claim of 1, wherein the kit comprises:
a) lidocaine hydrochloride oral topical solution with a 2% concentration;
b) a third container of mouthwash;
c) a plurality of glycerin swabs;
d) a plurality of buccal swabsticks;
e) a sterile urine cup;
f) a pair of gloves;
g) a face mask; and
h) a CSR (Central Supply Room) wrap;

13. A kit comprising a first container and following components placed therein:
a) a first tool for collection of cells for DNA (Deoxyribonucleic acid) testing from a person's buccal area;
b) a local anesthetic; and
c) a soothing agent.

14. The kit of claim 13, wherein the first tool is one or more buccal swabsticks.

15. The kit of claim 13, wherein the local anesthetic is lidocaine hydrochloride.

16. The kit of claim 13, wherein the soothing agent is one or more of glycerin swabs.

17. The kit of claim 13, further comprising one or more items for cleaning the buccal area.

18. The kit of claim 17, wherein the one or more components for cleaning the buccal area is selected from the group consisting of one or more of mouthwash, floss, toothbrush, and combinations thereof.

19. The kit of claim 13, further comprising one or more of gloves, face mask and CSR (Central Supply Room) wrap.

20. The kit of claim 13, wherein the kit comprises:
a) lidocaine Hydrochloride;
b) mouthwash;
c) a glycerin swab;
d) a buccal swabstick;
e) a toothbrush;
f) a pair of gloves;
g) a face mask; and
h) a csr (Central Supply Room) wrap.

21. The kit of claim 13, wherein the kit comprises:
a) lidocaine hydrochloride oral topical solution with a 2% concentration;
b) mouthwash;
c) a plurality of oral glycerin swabs;
d) a plurality of buccal swabsticks;
e) a toothbrush;
f) a pair of gloves;
g) a face mask; and
h) a CSR (Central Supply Room) wrap.

22. The kit of claim 13, wherein the kit comprises:
a) Lidocaine HCl;
b) a glycerin swab;
c) a buccal swabstick;
d) a pair of gloves;
e) a face mask; and
f) a CSR (Central Supply Room) wrap.

23. The kit of claim 13, wherein the kit comprises:
a) lidocaine hydrochloride oral topical solution with a 2% concentration;
b) a plurality of oral glycerin swabs;
c) a plurality of buccal swabsticks;
d) a pair of gloves;
e) a face mask; and
f) a CSR (Central Supply Room) wrap.
